SEQUENCE LISTING (27 sequences)

SEQ ID NO: 1 - protein, 354 aa, Homo sapiens
  REGION 1..354: note = UniProtKB-Q7Z3B1 (NEGR1_HUMAN)
MDMMLLVQGACCSNQWLAAVLLSLCCLLPSCLPAGQSVDFPWAAVDNMMVRKGDTAVLRCYLEDGASKGAWLNRSSIIFAGGDKWSVDPRVSISTLNKRDYSLQIQNVDVTDDGPYTCSVQTQHTPRTMQVHLTVQVPPKIYDISNDMTVNEGTNVTLTCLATGKPEPSISWRHISPSAKPFENGQYLDIYGITRDQAGEYECSAENDVSFPDVRKVKVVVNFAPTIQEIKSGTVTPGRSGLIRCEGAGVPPPAFEWYKGEKKLFNGQQGIIIQNFSTRSILTVTNVTQEHFGNYTCVAANKLGTTNASLPLNPPSTAQYGITGSADVLFSCWYLVLTLSSFTSIFYLKNAILQ

SEQ ID NO: 2 - protein, 348 aa, Mus musculus
  REGION 1..348: note = UniProtKB-Q80Z24 (NEGR1_MOUSE)
MVLLAQGACCSNQWLAAVLLSLCSCLPAGQSVDFPWAAVDNMLVRKGDTAVLRCYLEDGASKGAWLNRSSIIFAGGDKWSVDPRVSISTLNKRDYSLQIQNVDVTDDGPYTCSVQTQHTPRTMQVHLTVQVPPKIYDISNDMTINEGTNVTLTCLATGKPEPVISWRHISPSAKPFENGQYLDIYGITRDQAGEYECSAENDVSFPDVKKVRVIVNFAPTIQEIKSGTVTPGRSGLIRCEGAGVPPPAFEWYKGEKRLFNGQQGIIIQNFSTRSILTVTNVTQEHFGNYTCVAANKLGTTNASLPLNPPSTAQYGITGSACDLFSCWSLALTLSSVISIFYLKNAILQ

SEQ ID NO: 3 - protein, 329 aa, Mus musculus
  REGION 1..329: note = UniProtKB - A0A4W9 (A0A4W9_MOUSE)
MVLLAQGACCSNQWLAAVLLSLCSCLPAGQSVDFPWAAVDNMLVRKGDTAVLRCYLEDGASKGAWLNRSSIIFAGGDKWSVDPRVSISTLNKRDYSLQIQNVDVTDDGPYTCSVQTQHTPRTMQVHLTVQVPPKIYDISNDMTINEGTNVTLTCLATGKPEPVISWRHISPSAKPFENGQYLDIYGITRDQAGEYECSAENDVSFPDVKKVRVIVNFAPTIQEIKSGTVTPGRSGLIRCEGAGVPPPAFEWYKGEKRLFNGQQGIIIQNFSTRSILTVTNVTQEHFGNYTCVAANKLGTTNASLPLNQSSIPWQVFFMLKVSFLLVCIL

SEQ ID NO: 4 - protein, 122 aa, Mus musculus
  REGION 1..122: note = UniProtKB - H3BKU7 (H3BKU7_MOUSE)
MVLLAQGACCSNQWLAAVLLSLCSCLPAGQSVDFPWAAVDNMLVRKGDKWSVDPRVSISTLNKRDYSLQIQNVDVTDDGPYTCSVQTQHTPRTMQVHLTVQVPPKIYDISNDMTINEGTNVT

SEQ ID NO: 5 - protein, 329 aa, Mus musculus
  REGION 1..329: note = UniProtKB - D3Z4T6 (D3Z4T6_MOUSE)
MVLLAQGACCSNQWLAAVLLSLCSCLPAGQSVDFPWAAVDNMLVRKGDTAVLRCYLEDGASKGAWLNRSSIIFAGGDKWSVDPRVSISTLNKRDYSLQIQNVDVTDDGPYTCSVQTQHTPRTMQVHLTVQVPPKIYDISNDMTINEGTNVTLTCLATGKPEPVISWRHISPSAKPFENGQYLDIYGITRDQAGEYECSAENDVSFPDVKKVRVIVNFAPTIQEIKSGTVTPGRSGLIRCEGAGVPPPAFEWYKGEKRLFNGQQGIIIQNFSTRSILTVTNVTQEHFGNYTCVAANKLGTTNASLPLNLWCHLQMCWAHSWQCCLDILQA

SEQ ID NO: 6 - protein, 6 aa, synthetic construct
  REGION 1..6: note = minimum active sequence
GDKWSV

SEQ ID NO: 7 - protein, 6 aa, synthetic construct
  REGION 1..6: note = variant minimum active sequence
GDKISV

SEQ ID NO: 8 - protein, 96 aa, synthetic construct
  REGION 1..96: note = PepA
VDFPWAAVDNMLVRKGDTAVLRCYLEDGASKGAWLNRSSIIFAGGDKWSVDPRVSISTLNKRDYSLQIQNVDVTDDGPYTCSVQTQHTPRTMQVHL

SEQ ID NO: 9 - protein, 33 aa, synthetic construct
  REGION 1..33: note = PepA-1
DGASKGAWLNRSSIIFAGGDKWSVDPRVSISTL

SEQ ID NO: 10 - protein, 59 aa, synthetic construct
  REGION 1..59: note = PepA-2
VDFPWAAVDNMLVRKGDTAVLRCYLEDGASKGAWLNRSSIIFAGGDKWSVDPRVSISTL

SEQ ID NO: 11 - protein, 70 aa, synthetic construct
  REGION 1..70: note = PepA-3
DGASKGAWLNRSSIIFAGGDKWSVDPRVSISTLNKRDYSLQIQNVDVTDDGPYTCSVQTQHTPRTMQVHL

SEQ ID NO: 12 - protein, 96 aa, synthetic construct
  REGION 1..96: note = PepA variant
VDFPWAAVDNMLVRKGDTAVLRCYLEDGASKGAWLNRSSIIFAGGDKISVDPRVSISTLNKRDYSLQIQNVDVTDDGPYTCSVQTQHTPRTMQVHL

SEQ ID NO: 13 - protein, 33 aa, synthetic construct
  REGION 1..33: note = PepA-1 variant
DGASKGAWLNRSSIIFAGGDKISVDPRVSISTL

SEQ ID NO: 14 - protein, 59 aa, synthetic construct
  REGION 1..59: note = PepA-2 variant
VDFPWAAVDNMLVRKGDTAVLRCYLEDGASKGAWLNRSSIIFAGGDKISVDPRVSISTL

SEQ ID NO: 15 - protein, 70 aa, synthetic construct
  REGION 1..70: note = PepA-3 variant
DGASKGAWLNRSSIIFAGGDKISVDPRVSISTLNKRDYSLQIQNVDVTDDGPYTCSVQTQHTPRTMQVHL

SEQ ID NO: 16 - protein, 82 aa, synthetic construct
  REGION 1..82: note = PepB
PKIYDISNDMTINEGTNVTLTCLATGKPEPVISWRHISPSAKPFENGQYLDIYGITRDQAGEYECSAENDVSFPDVKKVRVI

SEQ ID NO: 17 - protein, 88 aa, synthetic construct
  REGION 1..88: note = PepC
APTIQEIKSGTVTPGRSGLIRCEGAGVPPPAFEWYKGEKRLFNGQQGIIIQNFSTRSILTVTNVTQEHFGNYTCVAANKLGTTNASLP

SEQ ID NO: 18 - protein, 33 aa, synthetic construct
  REGION 1..33: note = scramble
DGGSKGGFADLSSIIWARWNKARVDPLVSISTS

SEQ ID NO: 19 - protein, 33 aa, synthetic construct
  REGION 1..33: note = Scramble-GDKISV
NRGDKISVSVSSSSGGRAWDWDIAKPGLAILTF

SEQ ID NO: 20 - protein, 33 aa, synthetic construct
  REGION 1..33: note = PepA-1-6aa scramble
DGASKGAWLNRSSIIFAGVWGSKDDPRVSISTL

SEQ ID NO: 21 - DNA, 99 bp, synthetic construct
  misc_feature 1..99: note = PepA-1 coding sequence
  source 1..99: mol_type = other DNA
gacggcgccagcaagggcgcttggctgaaccggagcagcatcatcttcgccggcggagataagtggtccgtggaccctagagtgtctatcagcaccctg

SEQ ID NO: 22 - DNA, 99 bp, synthetic construct
  misc_feature 1..99: note = PepA-1 variant coding sequence
  source 1..99: mol_type = other DNA
gacggcgccagcaagggcgcttggctgaaccggagcagcatcatcttcgccggcggagataagatctctgtggaccctagagtgtccatcagcaccctg

SEQ ID NO: 23 - DNA, 54 bp, synthetic construct
  misc_feature 1..54: note = Leader sequence
  source 1..54: mol_type = other DNA
atgaagtgggtgaccttcatcagcctgctgtttctgttcagctccgcctactct

SEQ ID NO: 24 - DNA, 153 bp, synthetic construct
  misc_feature 1..153: note = leader sequence + PepA-1
  source 1..153: mol_type = other DNA
atgaagtgggtgaccttcatcagcctgctgtttctgttcagctccgcctactctgacggcgccagcaagggcgcttggctgaaccggagcagcatcatcttcgccggcggagataagtggtccgtggaccctagagtgtctatcagcaccctg

SEQ ID NO: 25 - DNA, 153 bp, synthetic construct
  misc_feature 1..153: note = leader sequence + PepA-1 variant
  source 1..153: mol_type = other DNA
atgaagtgggtgaccttcatcagcctgctgtttctgttcagctccgcctactctgacggcgccagcaagggcgcttggctgaaccggagcagcatcatcttcgccggcggagataagatctctgtggaccctagagtgtccatcagcaccctg

SEQ ID NO: 26 - protein, 51 aa, synthetic construct
  REGION 1..51: note = leader sequence + PepA-1
MKWVTFISLLFLFSSAYSDGASKGAWLNRSSIIFAGGDKWSVDPRVSISTL

SEQ ID NO: 27 - protein, 51 aa, synthetic construct
  REGION 1..51: note = leader sequence + PepA-1 variant
MKWVTFISLLFLFSSAYSDGASKGAWLNRSSIIFAGGDKISVDPRVSISTL

(19) United States
(12) Patent Application Publication
(10) Pub. No.: US 2025/0049888 A1
(43) Pub. Date: Feb. 13, 2025

(54) USE OF THE NEGR1 PROTEIN AND BIOLOGICALLY ACTIVE FRAGMENTS THEREOF IN THE THERAPEUTIC TREATMENT OF ALK-RELATED DISEASES

(71) Applicants: FONDAZIONE TELETHON ETS (70%), ROMA (IT); UNIVERSITA' DEGLI STUDI DI TRENTO (30%), Trento (IT)

(72) Inventors: Giovanni Piccoli, Povo (Trento) (IT); Francesca Pischedda, Povo (Trento) (IT)

(73) Assignees: FONDAZIONE TELETHON ETS (70%), Roma (IT); UNIVERSITA' DEGLI STUDI DI TRENTO (30%), Trento (IT)

(21) Appl. No.: 18/720,686

(22) PCT Filed: Dec. 21, 2022
(86) PCT No.: PCT/EP2022/087229

§ 371 Date: Jun. 17, 2024

(30) Foreign Application Priority Data
Dec. 22, 2021 (IT) 102021000032243

(51) Int. Cl. A61K 38/18; A61K 45/06; A61P 25/28; A61P 35/00

(52) U.S. Cl. CPC A61K 38/18; A61K 45/06; A61P 25/28; A61P 35/00

(57) ABSTRACT

The invention relates to novel Negr1 protein fragments having ALK-inhibiting or ALK-reducing activity and to the nucleic acids encoding therefor, as well as to the use of the Negr1 protein, its fragments and the nucleic acids encoding therefor in the therapeutic treatment of ALK-related diseases such as cancer and tauopathies.

The present invention is in the field of biopharmaceuticals.

In particular, the invention relates to Negr1 protein-based biopharmaceuticals and their use in the treatment of ALK-related diseases.

Negr1 (Neuronal growth regulator 1) belongs to the immunoglobulin superfamily (IgLON family), which are membrane exposed adhesion proteins. Negr1 contains 3 N-glycosylated C2-type Ig-like (immunoglobulin-like) domains with a N-terminal signal sequence. A putative GPI-attachment site is present close to the C-terminal end of the protein.

Negr1 is expressed at a high level in the brain, and its expression gradually increases during postnatal brain development, reaching a steady-state level in adulthood (Miyata et al. 2003). Negr1 expression regulates synapse formation of hippocampal neurons, neurite outgrowth, and dendritic spine plasticity (Pischedda et al. 2014; Pischedda and Piccoli 2015). Negr1 controls the formation of the upper layers in the cortical areas and the establishment of proper cortex-hippocampus connectivity via a functional and physical interaction with FGFR2 (Pischedda et al., 2014; Pischedda and Piccoli, 2015; Szczurkowska et al., 2018).

Negr1 has been identified as a commonly down-regulated gene in many types of human cancer tissues (Bobyn et al. 2020; Kim et al. 2014). For example, it has been observed that the expression of the IgLON family is altered in sporadic epithelial ovarian tumors and in metastatic neuroblastomas (Takita et al. 2011 and Mckie et al. 2012), indicating a possible tumor suppressor role for these genes.

Neuroblastoma (ORPHA: 635) represents about 10% of solid tumors in infants and children under the age of 15, with an annual incidence of about 1/70,000 in this age group.

Neuroblastoma can affect the sympathetic nervous system tissue, such as the paraspinal ganglia or adrenal medulla, and generate a noticeable mass in the chest, neck, pelvis, and/or abdomen (Mahapatra and Challagundla 2021).

Neuroblastoma harbors a variety of genetic changes, including a high frequency of MYCN amplification, loss of heterozygosity at 1p36 and 11q, and gain of genetic material from 17q. The anaplastic lymphoma kinase (ALK), initially identified as a fusion kinase in a subtype of non-Hodgkin's lymphoma (NPM-ALK) and adenocarcinoma of the lung (EML4-ALK), is also a frequent target of genetic alteration in advanced neuroblastoma and other cancers. ALK and MYCN genes localize in close proximity on chromosome 2p. Thus, amplification of the MYCN locus can also involve the ALK gene. Furthermore, ALK promotes MYCN transcription (Schönherr et al. 2012), and ALK is a direct transcriptional target of MYCN (Hasan et al. 2013). Such a positive feedback loop supports sustained tumor growth (Janoueix-Lerosey et al. 2008).

Clinically, neuroblastoma is classified into low- and high-risk. Risk groups are used to help in predicting the probability that a neuroblastoma patient will be cured and therefore how intensive his/her treatment should be. For example, a patient is classified in the low-risk group if it is expected that he/she can be cured with limited treatment, such as surgery alone. Instead, higher-risk patients are those who are likely to need more intensive treatment. The risk groups are based on the stage (extent) of the cancer, as well as other factors that can affect prognosis, the main one being age. The 5-year survival rate in low-risk patients is more than 95%, but it drops to 40% in high-risk patients (Bhoopathi et al. 2021).

Surgical intervention, alone or in combination with minimal chemotherapy treatment, can increase survival in low-risk cases. However, neuroblastoma in advanced stages is one of the most intractable pediatric cancers, even with recent therapeutic advances. A therapeutic strategy encompassing multimodal treatments may improve the prognosis for high-risk patients, although Stage III and IV patients (classification according to The International Neuroblastoma Staging System, INSS) often have a relapse that occurs early after the chemotherapy. Such relapse of tumors is often associated with more aggressive growth, resistance to chemotherapy, and metastasis (Speleman et al. 2016).

The long-term survival in patients with metastatic neuroblastoma is poor, partly due to the abundance of non-proliferating tumour cells. Surgical complication rates in patients with neuroblastoma range from 5-25%, depending on the stage of the tumour. Late effects, which can have diverse and devastating manifestations, should be considered. Chemotherapeutic regimens used to treat neuroblastoma may result in long-term toxicities, including cardiopulmonary toxicities (anthracyclines), ototoxicity (cisplatin), renal failure (ifosfamide and cisplatin), infertility and impotency (alkylating agents and radiation therapy), secondary cancers, and psychological effects.

There is therefore a need to identify novel therapeutic agents effective in the treatment of neuroblastoma. However, this need is not limited to neuroblastoma alone, but also extends to virtually all types of tumors, since tumors are known to possess the ability to develop resistance to traditional therapies, and the increased prevalence of drug-resistant tumors necessitates constant research to provide new treatments. It is also known that different patients respond differently to anticancer therapies, depending on many factors including the biological characteristics of the specific patient's tumor. This also contributes to the need for continuous research, in order to develop new and differentiated antitumor agents.

The prior art teaches that transient expression of Negr1 (by in vitro cell transfection of Negr1 expression vector) inhibits cells growth in various neuroblastoma lines (Takita et al. 2011). Kim et al. 2014 show that Negr1 overexpression in the human ovarian cancer cell line SKOV-3 results in attenuation of the oncogenic phenotype.

However, the aforementioned publications are research papers that relate to gene expression profiles in tumor tissues and that do not address the problem of providing a therapy that is concretely feasible and effective on cancer patients.

In order to overcome the shortcomings and drawbacks of the prior art, the present inventors first studied the effect of ectopic treatment of a variety of cancer cell lines with a soluble form of full-length Negr1 protein and found that Negr1 is capable of significantly reducing cancer cell proliferation in vitro and tumor volume ex vivo. This finding is unexpected, as the prior art does not teach or suggest that treatment with a soluble form of Negr1 protein would actually be feasible and effective in halting cancer growth.

The inventors then dissected the detected antitumor activity of the full-length Ngr1 protein and identified the responsible domain, i.e., the I/PepA (aa 1-127) domain of human Negr1. This led the inventors to identify an amino acid stretch within the full-length Negr1 protein, which is the minimum active sequence required for antitumor activity. This result is extremely advantageous, as it allows to provide a variety of Negr1 active fragments that retain the antitumor activity of the full-length protein. Accordingly, the expression “Negr1 active fragment(s)” or more simply “active fragment(s) indicates isolated peptide or polypeptide fragment(s) of a Negr1 protein which is/are capable of reducing ALK protein expression levels.

In some respects, peptide or polypeptide fragments may be advantageous over the whole protein, as the preparation of a full-length protein may require complex and costly biological procedures, while smaller fragments can be prepared by peptide synthesis. Additionally, the lower molecular weight of peptide or polypeptide fragments could result in improved activity and biodistribution, considering that the full length human Negr1 protein has a molecular weight of 38,719 Da in the native form and of above 50 kDa upon glycosylation.

The inventors also found that the antitumor activity of Negr1 and its active fragments correlates with their ability to reduce ALK protein expression levels in the treated cells.

Mutant forms of ALK are known to be implicated not only in neuroblastoma, but also across a range of other human tumor types including, inter alia, thyroid and renal cancers. Additionally, oncogenic ALK gene translocations or inversions are found in non-small cell lung cancer (NSCLC), anaplastic large-cell lymphoma (ALCL), inflammatory myofibroblastic tumor (IMT) and rare cases of other solid tumors (Villa et al., 2021).

It is also known that ALK drives tau phosphorylation and aggregation (Park et al., 2021), and that pathological tau aggregation occurs in Alzheimer's disease and in several other tauopathies including, inter alia, postencephalitic parkinsonism, chronic traumatic encephalopathy, Parkinson-dementia complex of Guam, and FTDP-17 due to MAPT mutations.

It is therefore envisaged that the Negr1 protein and its biologically active fragments shall also be effective in the treatment of tauopathies, e.g. neurodegenerative disorders characterized by the deposition of abnormal tau protein in the brain.

Accordingly, one aspect of the present invention is an isolated Negr1 protein or a biologically active fragment thereof of at least 6 amino acids in length, wherein the Negr1 biologically active fragment has the ability to reduce ALK protein expression levels, for use in the therapeutic treatment of a disease selected from the group consisting of tumor diseases and a tauopathies.

In the present description, the expression “a Negr1 protein” includes, inter alia, the human Negr1 protein as well as its orthologs from other animal species such as mouse.

The amino acid sequences of the predominant isoforms of human and mouse Negr1 are available from the UniProt Databese under accession numbers Q7Z3B1 and Q80Z24, respectively.

UniProtKB-Q7Z3B1 (NEGR1_HUMAN)
(SEQ ID NO: 1)
MDMMLLVQGACCSNQWLAAVLLSLCCLLPSCLPAGQSVDFPWAAVDNMM
VRKGDTAVLRCYLEDGASKGAWLNRSSIIFAGGDKWSVDPRVSISTLNK
RDYSLQIQNVDVTDDGPYTCSVQTQHTPRTMQVHLTVQVPPKIYDISND
MTVNEGTNVTLTCLATGKPEPSISWRHISPSAKPFENGQYLDIYGITRD
QAGEYECSAENDVSFPDVRKVKVVVNFAPTIQEIKSGTVTPGRSGLIRC
EGAGVPPPAFEWYKGEKKLFNGQQGIIIQNFSTRSILTVTNVTQEHFGN
YTCVAANKLGTTNASLPLNPPSTAQYGITGSADVLFSCWYLVLTLSSFT
SIFYLKNAILQ
UniProtKB-Q80Z24 (NEGR1_MOUSE)
(SEQ ID NO: 2)
MVLLAQGACCSNQWLAAVLLSLCSCLPAGQSVDFPWAAVDNMLVRKGDT
AVLRCYLEDGASKGAWLNRSSIIFAGGDKWSVDPRVSISTLNKRDYSLQ
IQNVDVTDDGPYTCSVQTQHTPRTMQVHLTVQVPPKIYDISNDMTINEG
TNVTLTCLATGKPEPVISWRHISPSAKPFENGQYLDIYGITRDQAGEYE
CSAENDVSFPDVKKVRVIVNFAPTIQEIKSGTVTPGRSGLIRCEGAGVP
PPAFEWYKGEKRLFNGQQGIIIQNFSTRSILTVTNVTQEHFGNYTCVAA
NKLGTTNASLPLNPPSTAQYGITGSACDLFSCWSLALTLSSVISIFYLK
NAILQ

Further isoforms of the mouse Negr1 protein are available from the UniProt Database under accession numbers A0A4W9, H3BKU7 and D3ZAT6.

UniProtKB-A0A4W9 (A0A4W9_MOUSE)
(SEQ ID NO: 3)
MVLLAQGACCSNQWLAAVLLSLCSCLPAGQSVDFPWAAVDNMLVRKGDT
AVLRCYLEDGASKGAWLNRSSIIFAGGDKWSVDPRVSISTLNKRDYSLQ
IQNVDVTDDGPYTCSVQTQHTPRTMQVHLTVQVPPKIYDISNDMTINEG
TNVTLTCLATGKPEPVISWRHISPSAKPFENGQYLDIYGITRDQAGEYE
CSAENDVSFPDVKKVRVIVNFAPTIQEIKSGTVTPGRSGLIRCEGAGVP
PPAFEWYKGEKRLFNGQQGIIIQNFSTRSILTVTNVTQEHFGNYTCVAA
NKLGTTNASLPLNQSSIPWQVFFMLKVSFLLVCIL
UniProtKB-H3BKU7 (H3BKU7_MOUSE)
(SEQ ID NO: 4)
MVLLAQGACCSNQWLAAVLLSLCSCLPAGQSVDFPWAAVDNMLVRKGDK
WSVDPRVSISTLNKRDYSLQIQNVDVTDDGPYTCSVQTQHTPRTMQVHL
TVQVPPKIYDISNDMTINEGTNVT
UniProtKB-D3Z4T6 (D3Z4T6_MOUSE)
(SEQ ID NO: 5)
MVLLAQGACCSNQWLAAVLLSLCSCLPAGQSVDFPWAAVDNMLVRKGDT
AVLRCYLEDGASKGAWLNRSSIIFAGGDKWSVDPRVSISTLNKRDYSLQ
IQNVDVTDDGPYTCSVQTQHTPRTMQVHLTVQVPPKIYDISNDMTINEG
TNVTLTCLATGKPEPVISWRHISPSAKPFENGQYLDIYGITRDQAGEYE
CSAENDVSFPDVKKVRVIVNFAPTIQEIKSGTVTPGRSGLIRCEGAGVP
PPAFEWYKGEKRLFNGQQGIIIQNFSTRSILTVTNVTQEHFGNYTCVAA
NKLGTTNASLPLNLWCHLQMCWAHSWQCCLDILQA

The expression “a Negr1 protein” further includes a variant protein derived from any one of the above-identified human and mouse Negr1 proteins by substitution of one or more amino acid residues, and which is at least 90% identical to any one of the above-identified human and mouse Negr1 proteins.

In making amino acid substitutions, generally the amino acid residue to be substituted can be a conservative amino acid substitution, for example, a polar residue is substituted with a polar residue, a hydrophilic residue with a hydrophilic residue, hydrophobic residue with a hydrophobic residue, a positively charged residue with a positively charged residue, or a negatively charged residue with a negatively charged residue.

Since all the amino acid sequences in the UniProt Database that show an identity % of at least 90% to human Negr1 are Neuronal growth regulator 1 (Negr1) proteins, it is envisaged that variant proteins that are at least 90% identical to SEQ ID NO:1, SEQ ID NO:2, SEQ ID NO: 3, SEQ ID NO:4 or SEQ ID NO:5, shall retain the ability to reduce ALK protein expression levels.

The comparison of sequences and determination of percent identity between two sequences is accomplished using a mathematical algorithm such as the blastp (blastp BLASTP 2.9.0+) program, using the blosum62 matrix.

Accordingly, a preferred embodiment of the invention is an isolated Negr1 protein or a biologically active fragment thereof of at least 6 amino acids in length for use in the therapeutic treatment of a tumor disease or a tauopathy, wherein the isolated Negr1 protein is selected from the group consisting of:
(i) amino acid sequences which comprise, consist essentially of or consist of SEQ ID NO:1, SEQ ID NO:2, SEQ ID NO:3, SEQ ID NO:4 or SEQ ID NO:5;
(ii) amino acid sequences which comprise, consist essentially of or consist of an amino acid sequence at least 90% identical to SEQ ID NO:1, SEQ ID NO:2, SEQ ID NO:3, SEQ ID NO: 4 or SEQ ID NO:5 and which are capable of reducing ALK protein expression levels.

The aforementioned % identity ranges include the following embodiments: at least 91%, at least 92%, at least 93%, at least 94%, at least 95%, at least 96%, at least 97%, at least 98%, and at least 99% to SEQ ID NO:1; at least 91%, at least 92%, at least 93%, at least 94%, at least 95%, at least 96%, at least 97%, at least 98%, and at least 99% to SEQ ID NO:2; at least 91%, at least 92%, at least 93%, at least 94%, at least 95%, at least 96%, at least 97%, at least 98%, and at least 99% to SEQ ID NO:3; at least 91%, at least 92%, at least 93%, at least 94%, at least 95%, at least 96%, at least 97%, at least 98%, and at least 99% to SEQ ID NO: 4; at least 91%, at least 92%, at least 93%, at least 94%, at least 95%, at least 96%, at least 97%, at least 98%, and at least 99% to SEQ ID NO:5.

As it will be illustrated in the experimental section below, the inventors found that SEQ ID NO: 6 is the minimum active sequence of the Negr1 amino acid sequences that sustains their biological activity of reducing ALK protein expression levels. The inventors also surprisingly found that a variant of the aforementioned minimum biologically active sequence, wherein W (tryptophan) is replaced by I (isoleucine) (SEQ ID NO:7), retains the ALK expression-reducing activity of SEQ ID NO:6.

Accordingly, another preferred embodiment of the invention is an isolated Negr1 protein or a biologically active fragment thereof for use in the therapeutic treatment of a tumor disease or a tauopathy, wherein the isolated Negr1 protein is selected from the group consisting of: (iii) amino acid sequences which comprise, consist essentially of or consist of an amino acid sequence at least 90% identical to SEQ ID NO:1, SEQ ID NO:2, SEQ ID NO:3, SEQ ID NO: 4 or SEQ ID NO:5 and which include the minimum biologically active sequence SEQ ID NO: 6 or SEQ ID NO:7.

To the inventors' knowledge, isolated peptides or polypeptides which are the biologically active fragments of a Negr1 protein capable of reducing ALK protein expression levels are not disclosed per se in the prior art.

Accordingly, the scope of the invention also encompasses an isolated peptide or polypeptide, which is a fragment of a Negr1 protein having a length of at least 6 amino acids and capable of reducing ALK protein expression levels, wherein the expression “a Negr1 protein” includes isoforms, orthologs and variant proteins as defined above.

Examples of such fragments contain 6 to 96 amino acids from a Negr1 protein amino acid sequence, wherein the expression “a Negr1 protein” includes isoforms, orthologs and variant proteins as defined above. Further examples of such fragments contain 6 to 35 amino acids, or 6 to 70 amino acids, or 35 to 70 amino acids from a Negr1 protein amino acid sequence as defined above. Specific examples are fragments which contain 6, 33, 70 or 96 amino acids from a Negr1 protein amino acid sequence as defined above.

Preferred embodiments of such fragments include the minimum biologically active sequence SEQ ID NO:6 or SEQ ID NO:7.

The most preferred embodiments of such fragments are: “PepA” of SEQ ID NO:8; “PepA-1” of SEQ ID NO:9; “PepA-2” of SEQ ID NO: 10; “PepA-3” of SEQ ID NO:11; the minimum biologically active sequence of SEQ ID NO:6; “PepA variant” of SEQ ID NO:12; “PepA-1 variant” of SEQ ID NO: 13; “PepA-2 variant” of SEQ ID NO:14; “PepA-3 variant” of SEQ ID NO: 15; and the minimum variant biologically active sequence of SEQ ID NO:7.

The peptides or polypeptides of the invention are advantageously characterized by a shorter amino acid sequence and lower steric hindrance as compared to the full length Negr1 protein, which may result in increased ease of synthesis and of penetration into cell membranes. Usually, peptide or polypeptide fragments also have high activity, specificity, and affinity; minimal drug-drug interaction and biological and chemical diversity. An added benefit of the peptides or polypeptides as drugs is that they usually do not accumulate in specific organs (e.g., kidney or liver), which can help to minimize their toxic side effects. Usually, they are also less immunogenic than recombinant antibodies or proteins.

Such features make the peptides or polypeptides of the invention particularly suitable for use as therapeutics although, as mentioned, the full-length Negr1 protein is also effective.

The scope of the invention also encompasses an isolated nucleic acid (DNA or RNA) comprising a nucleotide sequence coding for a Negr1 peptide or polypeptide as defined above, as well as its use in the therapeutic treatment of a tumor disease or a tauopathy.

Also within the scope of the invention is an expression vector (preferably a viral expression vector) comprising the aforementioned nucleotide sequence coding for a Negr1 peptide or polypeptide as defined above, as well as a host cell transformed with said expression vector.

By way of a non-limiting example, nucleotide sequences optimized for the expression in human cells of the 33 aa peptides “PepA-1” or “PepA-1 variant” are provided herein below:

(SEQ ID NO: 21)
GACGGCGCCAGCAAGGGCGCTTGGCTGAACCGGAGCAGCATCATCTTCG
CCGGCGGAGATAAGTGGTCCGTGGACCCTAGAGTGTCTATCAGCACCCT
G (PepA-1 nt sequence)
(SEQ ID NO: 22)
GACGGCGCCAGCAAGGGCGCTTGGCTGAACCGGAGCAGCATCATCTTCG
CCGGCGGAGATAAGATCTCTGTGGACCCTAGAGTGTCCATCAGCACCCT
G (PepA-1 variant nt sequence)

Upstream of the two sequences the leader sequence of Serum albumin pre proprotein NP_000468 may be inserted:

(SEQ ID NO: 23)
ATGAAGTGGGTGACCTTCATCAGCCTGCTGTTTCTGTTCAGCTCCGCCT
ACTCT (leader sequence of Serum albumin pre
proprotein NP_000468)

Based on the above nucleic acids, two expression vectors can be constructed each including one of the following exogenous nucleic acids:

(SEQ ID NO: 24)
ATGAAGTGGGTGACCTTCATCAGCCTGCTGTTTCTGTTCAGCTCCGCCT
ACTCTGACGGCGCCAGCAAGGGCGCTTGGCTGAACCGGAGCAGCATCAT
CTTCGCCGGCGGAGATAAGTGGTCCGTGGACCCTAGAGTGTCTATCAGC
ACCCTG (leader sequence + PepA-1)
(SEQ ID NO: 25)
ATGAAGTGGGTGACCTTCATCAGCCTGCTGTTTCTGTTCAGCTCCGCCT
ACTCTGACGGCGCCAGCAAGGGCGCTTGGCTGAACCGGAGCAGCATCAT
CTTCGCCGGCGGAGATAAGATCTCTGTGGACCCTAGAGTGTCCATCAGC
ACCCTG (leader sequence + PepA-1 variant),

encoding for the following peptides optimized for SEQ ID NO:24 encodes for a PepA-1 nd SEQ ID NO:5 encode for the following peptides optimized for expression in human cells:

(SEQ ID NO: 26)
MKWVTFISLLFLFSSAYSDGASKGAWLNRSSIIFAGGDKWSVDPRVSIS
TL (leader sequence + PepA-1)
(SEQ ID NO: 27)
MKWVTFISLLFLFSSAYSDGASKGAWLNRSSIIFAGGDKISVDPRVSIS
TL (leader sequence + PepA-1 variant).

The skilled in the art is able to design and manufacture further embodiments of the nucleic acids, expression vectors and host cells of the invention, based on the known nucleotide sequences encoding for Negr1 and using the common general knowledge available in the field of recombinant DNA technologies, without undue burden.

Insofar as the therapeutic applications are concerned, preferred tumor diseases to be treated are selected from the group consisting of neuroblastoma, ganglioglioma, gangliocytoma, adenocarcinoma of the lung, renal cell carcinoma, squamous cell esophageal carcinoma, breast cancer, thyroid cancer, colon adenocarcinoma, glioblastoma, squamous cell esophageal carcinoma, melanoma, ovarian cancer, non-small cell lung cancer (NSCLC), anaplastic large-cell lymphoma (ALCL) and inflammatory myofibroblastic tumor (IMT).

Preferred tauopathies to be treated are selected from the group consisting of Alzheimer's disease, postencephalitic parkinsonism, Chronic traumatic encephalopathy (CTE), Parkinson-dementia complex of Guam, Frontotemporal dementia and parkinsonism linked to chromosome 17 (FTDP-17), Primary age-related tauopathy (PART) dementia, Progressive supranuclear palsy (PSP), Corticobasal degeneration (CBD), Vacuolar tauopathy, Meningioangiomatosis, Subacute sclerosing panencephalitis (SSPE).

The Negr1 protein and biologically active fragments thereof may be formulated in a pharmaceutical composition comprising the active ingredient and one or more pharmaceutically acceptable excipient.

The person skilled in the art is able to select the administration route, pharmaceutical dosage form and dose of the active ingredient to be administered, depending on a number of factors including, inter alia, the specific disease to be treated and the characteristics of the patient.

The Negr1 protein and biologically active fragments thereof for use according to the invention may also be administered to a patient affected by tumor as a combined therapy, i.e. as combined simultaneous, separate or sequential administration with a compound or substance having anti-tumor activity or with a radiotherapy agent.

According to a preferred embodiment, the compound or substance having anti-tumor activity is selected from the group consisting of Cyclophosphamide, Cisplatin, carboplatin, Vincristine, Doxorubicin, Etoposide, Topotecan, Melphalan, Busulfan, Thiotepa, dinutuximab, dinutuximab beta, Crizotinib, Ceritinib, Alectinib, Brigatinib, Lorlatinib, Ensartinib, Entrectinib, and any combination thereof.

Similarly, the Negr1 protein and biologically active fragments thereof for use according to the invention may be administered to a patient affected by a tauopathy as a combined therapy, i.e. by combined simultaneous, separate or sequential administration with a compound or substance active in the therapeutic treatment of the tauopathy.

Further features and advantages of the present invention will become apparent from the following detailed description of the experiments carried out by the inventors, which is by making reference to the appended drawings, in which:

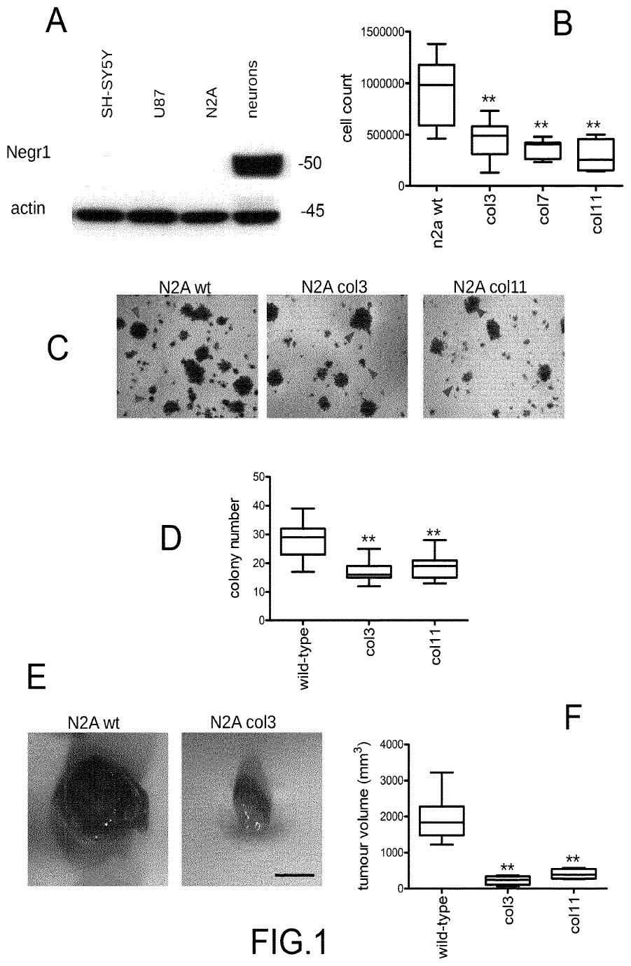

FIG. 1 illustrates the experimental results obtained by the inventors, which show that Negr1 over-expression halts neuroblastoma growth in vitro and in vivo. (A) Western-blot analysis of human neuroblastoma (SH5Y), glioblastoma (U87), murine neuroblastoma (N2a) and murine primary cortical neurons. Samples were stained with anti-Negr1 and anti-actin antibodies. (B) The proliferation rate of wild-type N2a cells as well as 3 different clones stably expressing Negr1 (colony 3, 7, and 11) was analyzed. N2a clones were seeded at a fixed amount (20,000 cells) at day 0. The cell number was counted at DIV5; n=12, **p<0.01 vs. wild-type. (C) 20,000 N2a wild-type or stably expressing Negr1 (col3 and 11) cells were seeded in soft-agar and cultured for 14 days. Next, cells were stained with crystal violet. (D) The graph reports number of colony (cluster encompassing more than 50 cells); n=6, **p<0.01 vs. wild-type. (E) 1 million N2a cells (wild-type, clone 3, and clone 11) were injected subcutaneously in CD1 immunodeficient nude mice. Tumor growth was monitored on day 3,5,7,9, 13 and 15 after injection. The images show two representative tumor masses, scale bar=1 cm. (F) The graph reports tumor volume; n=6, **p<0.01 vs. wild-type.

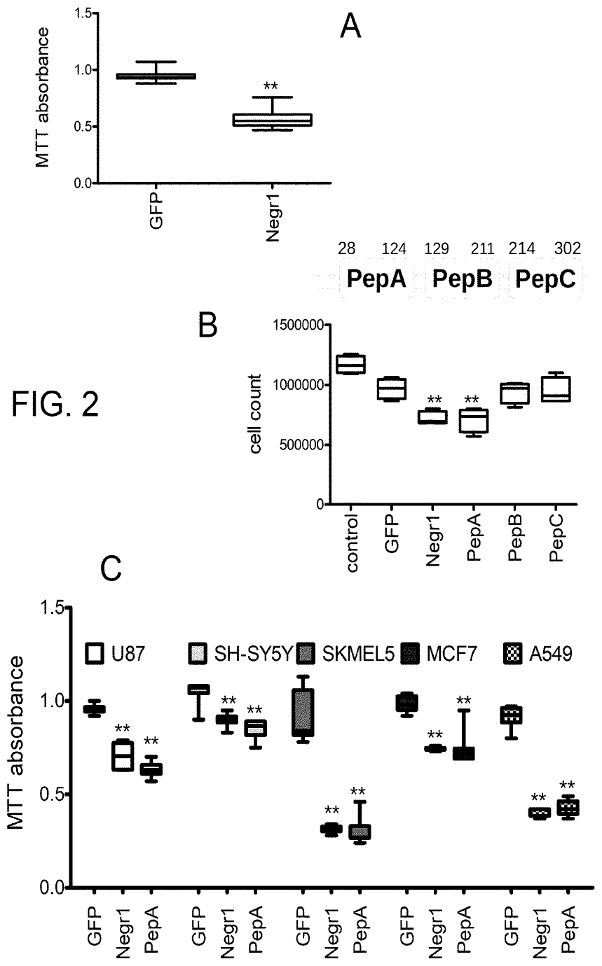

FIG. 2 illustrates the experimental results obtained by the inventors, which show that PepA, which is one embodiment of the invention, halts neuroblastoma growth in vitro. (A) The proliferation rate of wild-type N2a cells treated with recombinant GFP or Negr1 (200 ng, daily) was analyzed. N2a clones were seeded at fixed amount (20,000 cells) at day 0 and cultured for 5 days. Culture viability was measured by MTT assay. The graph reports cell viability expressed as fold-over not treated culture; n=10, **p<0.01 vs. GFP. (B) The cartoon depicts the boundaries of the Negr1 IgG-like domains, as reported in NCBI (NP_776169.2). The proliferation rate of wild-type N2a cells treated with recombinant GFP, Negr1 or Negr1-derived PepA, PepB, and PepC (200 ng, daily) was analysed. N2a cells were seeded at fixed amount (20,000 cells) at day 0 and cultured for 5 days. The graph reports cell number at day 5; n=12, **p<0.01 vs GFP. (C) The proliferation rate of U87, SK-MEL5, MCF7, A549, and SH-SY5Y cells treated with recombinant GFP, Negr1, or PepA (200 ng, daily) was analyzed. The cells were seeded at a fixed amount (20,000 cells) at day 0 and cultured for 5 days. Culture viability was measured by MTT assay. The graph reports cell viability expressed as fold over vehicle; n=8, **p<0.01 vs GFP.

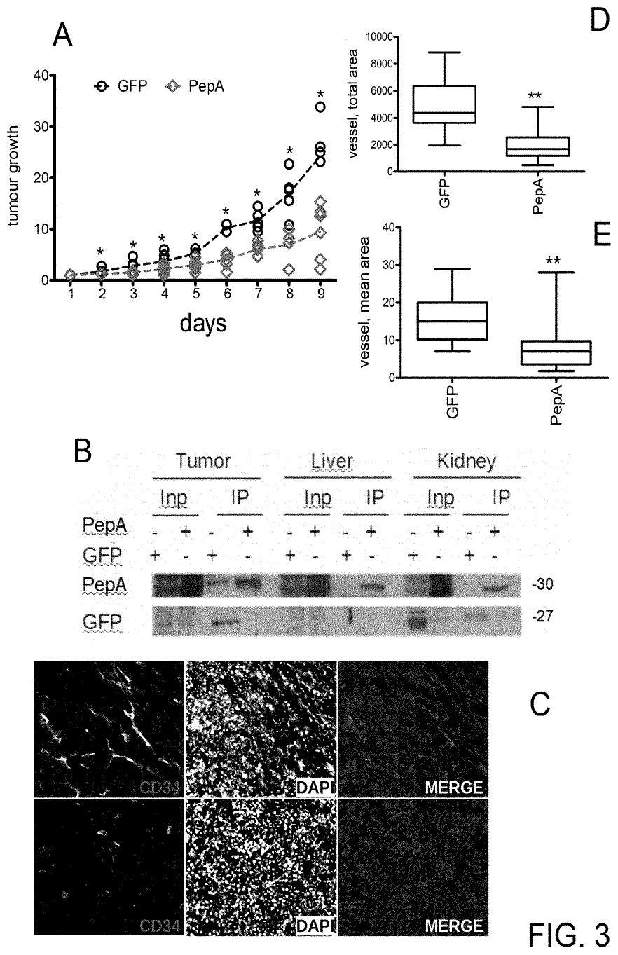

FIG. 3 illustrates the experimental results obtained by the inventors, which show that PepA halts neuroblastoma growth in vivo. (A) 1 million N2a cells were injected subcutaneously in CD1 immunodeficient nude mice. When tumor reached 100 mm³ volume, 2 μg of GFP or PepA were injected in situ. The treatment was repeated every second day. Tumor growth was monitored from day 1 to 9 after injection. The graph reports tumor volume growth expressed as fold-over day 1; n=9, **p<0.01 vs. GFP. (B) The presence of GFP and PepA in the tumor, liver, and kidney was investigated by STREP-pulldown. (C) 20 μm thick slices gathered from tumors treated with GFP or PepA were analysed by immunofluorescence. The specimen were stained with anti CD34 antibody (that decorates immature vessels, red) and DAPI (to visualize cell nucleus, blue). (D-E) The graphs report vessel number (E) and mean area (arbitrary unit) (E); n=4, *p<0.05 vs. GFP.

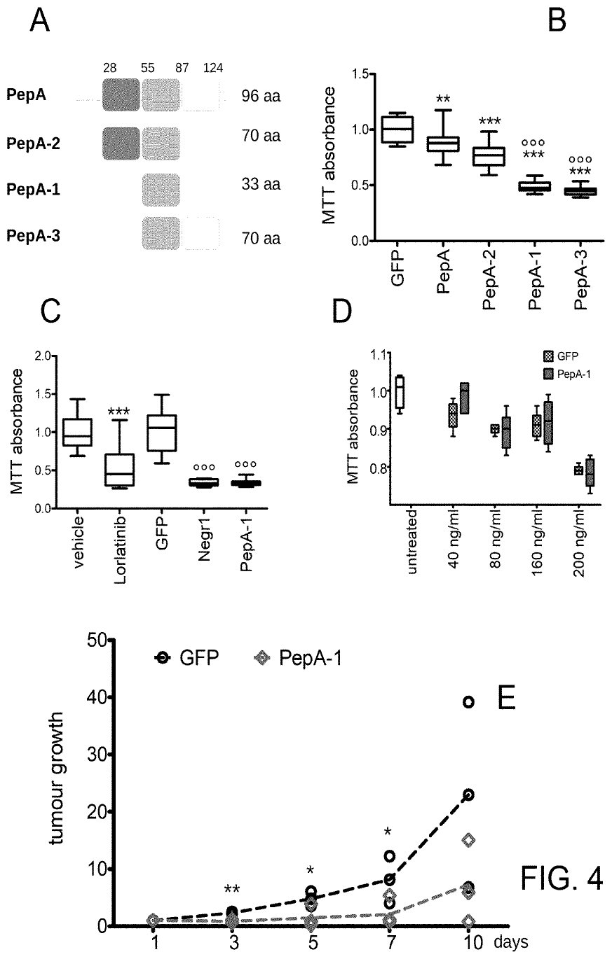

FIG. 4 illustrates the experimental results obtained by the inventors, which show that further soluble Negr1-derived peptides (i.e., PepA-1, PepA-2 and PepA-3) halt neuroblastoma growth in vitro and in vivo. (A) The cartoon depicts the boundaries of aforementioned peptides. (B) The proliferation rate of wild-type N2a cells treated with recombinant GFP, PepA, PepA-1, PepA-2, or PepA-3 (500 ng, daily) was analyzed. N2a cells were seeded at a fixed amount (20,000 cells) at day 0 and cultured for 5 days, and eventually measured by MTT assay. The graph reports cell vitality at day 5 normalized to GFP value; n=10, **p<0.01 vs. GFP. (C) The proliferation rate of wild-type N2a cells treated with vehicle, Lorlatinib, GFP, rNegr1, and PepA-1 (3 μM, daily) was analyzed. N2a cells were seeded at fixed amount (20,000 cells) at Day 0 and cultured for 5 days, and eventually measured by MTT assay. The graph reports cell vitality at day 5 normalized to values measured upon vehicle (for Lorlatinib) or GFP (for rNegr1, and PepA-1) treatment; n=10, ***p<0.01 vs vehicle, ∘∘∘p<0.01 vs GFP. (D) Primary cortical neurons were treated daily with 40-80-160-200 ng/ml of PepA-1 or GFP starting at DIV7. After 5 days, cellular toxicity was evaluated by MTT. The graph reports cell vitality normalized to GFP value; n=6. (E) 1 million N2a cells were injected subcutaneously in CD1 immunodeficient nude mice. When tumors reached 100 mm³ volume, 2 ug of GFP or PepA-1 were injected in situ. The treatment was repeated every second day. Tumor growth was monitored on day 3, 5, 7 and 10 after injection. The graph reports tumor volume growth expressed as fold-over day 1; n=3, *p<0.05 vs. GFP

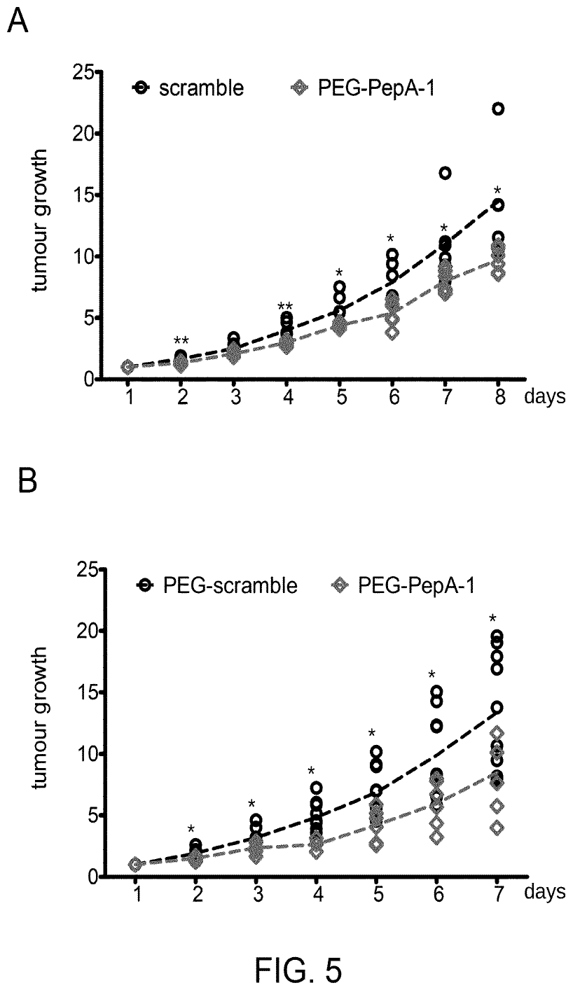

FIG. 5 illustrates the experimental results obtained by the inventors, which show that synthetic PEG-PepA-1 halts neuroblastoma growth in vitro and in vivo. (A) 1 million N2a cells were injected subcutaneously in CD1 immunodeficient nude mice. When tumors reached 100 mm³ volume, 10 μg of PEG-scramble peptide or PEG-PepA-1 were injected in situ. The treatment was repeated every second day. Tumor growth was monitored daily after injection. The graph reports tumor volume growth expressed as fold-over day 1; n=6, *p<0.05, **p<0.01 vs. PEG-scramble. (B) 1 million N2a cells were injected subcutaneously in CD1 immunodeficient nude mice. When tumors reached 100 mm³ volume, 150 μg of PEG-scramble peptide or PEG-PepA-1 were injected intraperitoneally. The treatment was repeated every second day. Tumor growth was monitored daily after injection. The graph reports tumor volume growth expressed as fold-over day 1; n=6-8, *p<0.05 vs. PEG-scramble.

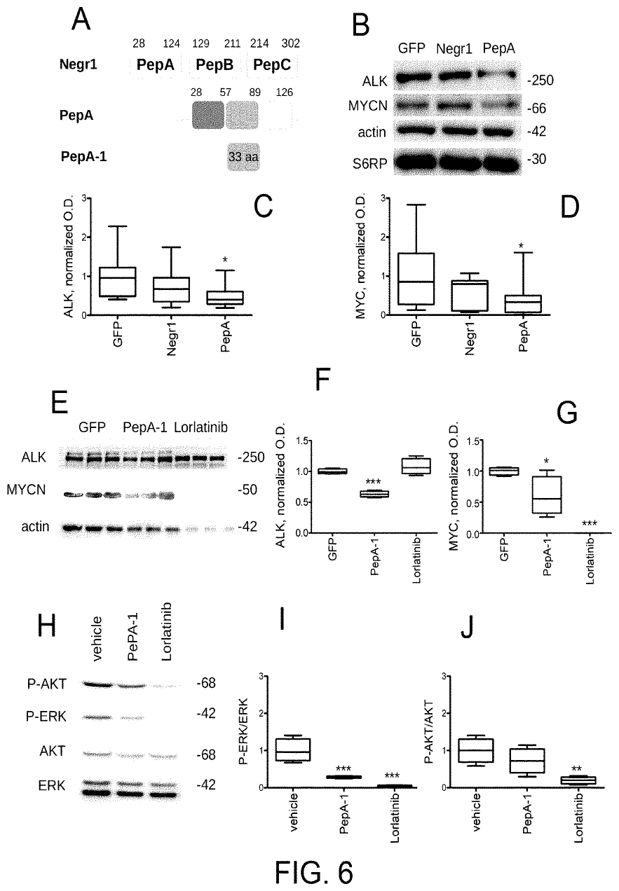

FIG. 6 illustrates the experimental results obtained by the inventors, which show that Peptide PepA modulates ALK signaling. (A) The cartoon depicts the boundaries of PepA, PepB and PepC. (B) 50,000 N2a cells were treated with 300 ng/ml GFP, rNegr1, or PepA daily for 5 days. At DIV5, the culture was processed for western blotting and the protein expression level was evaluated for ALK and MYCN as well as for actin and S6 ribosomal protein. (C-D) The graphs report ALK (C) and MYC (D) levels, normalized over actin and S6RP amount, and expressed as fold over cell treated with GFP; n=6, *p<0.05 vs. GFP. (E) 50,000 N2a cells were treated with 3 μM GFP, Lorlatinib or PepA-1 daily for 5 days. At DIV5, the culture was processed for western-blotting and the protein expression level was evaluated for ALK and MYCN as well as for actin and S6 ribosomal protein. (F-G) The graphs report ALK (F) and MYC (G) level, normalized over actin and S6RP amount, and expressed as fold-over cell treated with GFP; n=6, *p<0.05, ***p<0.001 vs. GFP. (H) 150,000 N2a cells were treated with 3 μM GFP, Lorlatinib or PepA-1 for 10 minutes. Next, the culture was processed for western blotting and the protein expression level was evaluated for AKT, phospho-AKT, ERK1/2 and phospho-ERK1/2, and for the S6 ribosomal protein. (I-J) The graphs report pAKT/AKT (I) and pERK/ERK (J) ratio expressed as fold over cell treated with vehicle; n=6, **p<0.01, ***p<0.001 vs. vehicle.

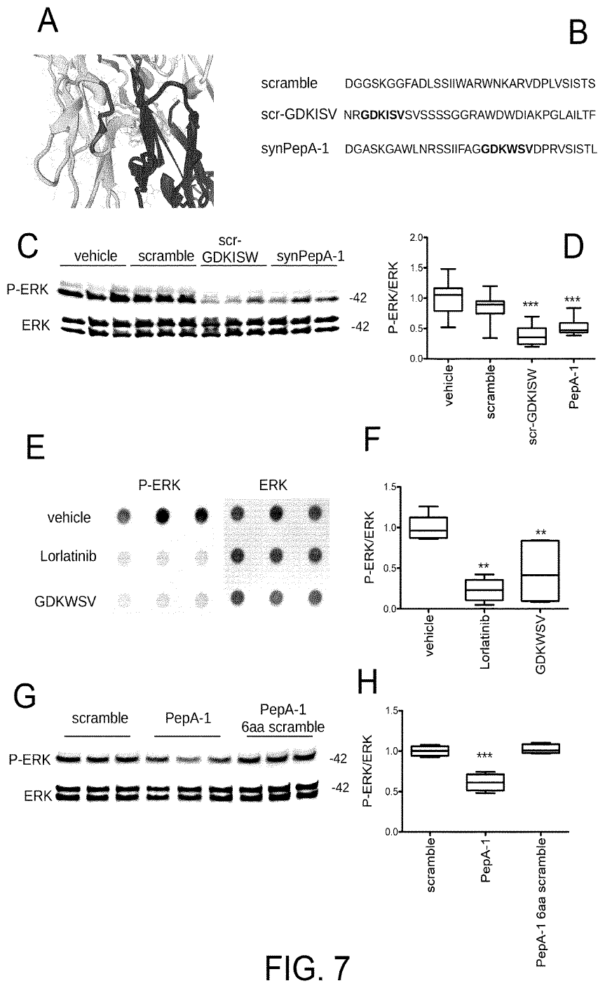

FIG. 7 illustrates the experimental results obtained by the inventors, which show that the synthetic version of PepA-1 and a scramble peptide containing a variant minimum biologically active sequence modulate ALK signaling. (A) The cartoon depicts the position of the minimum biologically active sequence GDKWSV (SEQ ID NO:8) (adapted from PDB: 6DLD). (B) The sequence of the synthetic peptides. (C) 150,000 N2a cells were treated with vehicle or the following synthetic peptides (10 μM, 10 minutes 10 μM): scramble peptide, scr-GDKISV a PepA-1 derived peptide but including the variant minimum biologically active sequence GDKISV (SEQ ID NO:9), and PepA-1. Next, the culture was processed for western blotting, and the expression level of ERK1/2 and phospho-ERK1/2 was evaluated. (D) The graph reports phospho-ERK/ERK ratio as fold over vehicle; n=8, *p<0.05, ***p<0.01 vs vehicle. (E) 150,000 N2a cells were treated with 3 or 30 μM vehicle, Lorlatinib or GDKWSV peptide for 10 minutes. Next, the culture was processed for dot-blotting, and the expression level of ERK1/2 and phospho-ERK1/2 was evaluated. (F) The graph reports phospho-ERK/ERK ratio as fold over vehicle; n=5, **p<0.01 vs vehicle. (G) 150,000 N2a cells were treated with the following synthetic peptides (10 μM, 10 minutes 10 μM): scramble peptide, PepA-1, and PepA-1 6aa scramble, a PepA-1 derived peptide but bearing a scramble sequence instead of the GDKISV esapeptide. Next, the culture was processed for western blotting, and the expression level of ERK1/2 and phospho-ERK1/2 was evaluated. (H) The graph reports phospho-ERK/ERK ratio as fold over vehicle; n=4, ***p<0.01 vs other conditions.

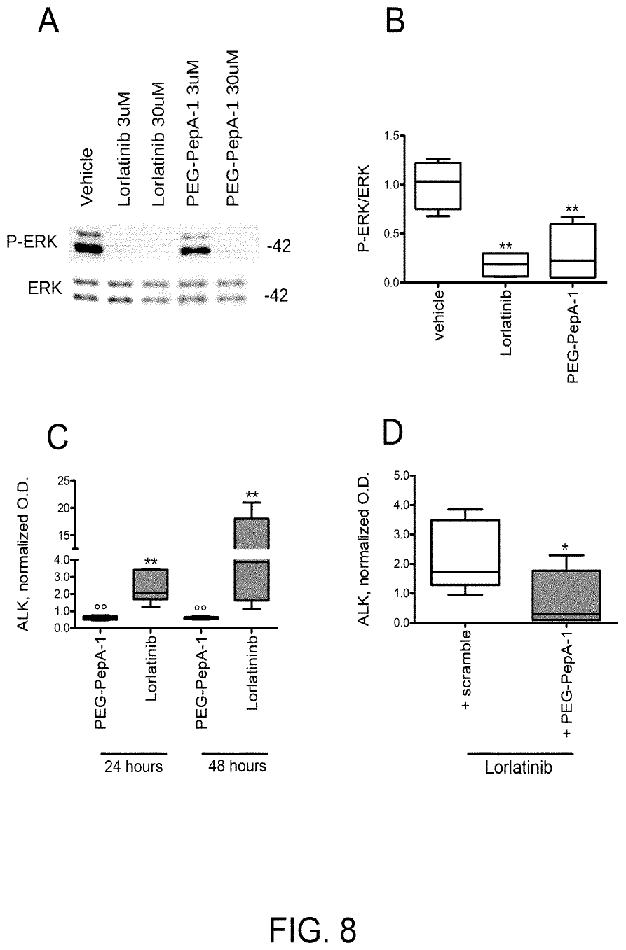

FIG. 8 illustrates the experimental results obtained by the inventors relating to the efficacy of a pegylated form of PepA-1, called PEG-PepA-1. A) 150,000 N2a cells were treated with 3 or 30 μM vehicle, Lorlatinib or PEG-PepA-1 for 10 minutes. Next, the culture was processed for western-blotting and the protein expression level was evaluated for ERK1/2 and phospho-ERK1/2. (B) The graph reports phospho-ERK/ERK ratio as fold-over vehicle upon 30 μM treatment; n=8, **p<0.01 vs. vehicle. (C) 150,000 N2a cells were treated with 5 μM vehicle, Lorlatinib, scramble peptide or PEG-PepA-1 for 24 or 48 hours. Next, the culture was processed for western-blotting and the protein expression level was evaluated for ALK. The graph reports the normalized ALK optical density as fold-over relative control; n=4, **p<0.01 vs. PEG-PepA-1, ∘∘ p<0.01 vs scramble peptide. (D) 150,000 N2a cells were treated with vehicle, Lorlatinib plus scramble peptide or plus PEG-PepA-1 for 24 hours at 5 μM. Next, the culture was processed for western-blotting and the protein expression level was evaluated for ALK. The graph reports the normalized ALK optical density as fold-over vehicle; n=4, **p<0.01 vs. scramble peptide.

The results obtained by the inventors are illustrated in detail below.

In a first set of experiments, the inventors observed that that Negr1 protein is downregulated in the brain cancer-derived cell lines SH5Y (human neuroblastoma), U87 (glioblastoma) and N2a (murine neuroblastoma). The inventors also found that ectopic Negr1 expression in N2a cells significantly reduced cell proliferation and that Negr1-expressing N2a cell clones had a severely reduced ability to generate colonies on soft agar. Additionally, in an in vivo study with the Xenograft mouse model, which represents an established tool to investigate cancer progression in vivo, they observed that the tumors derived from subcutaneous injection (s.c.) of Negr1-expressing N2a cells were significantly smaller in size than the tumors derived from wild-type N2a cells.

These findings demonstrate that Negr1 over-expression is capable of halting cancer growth both in vitro and in vivo.

In a second set of experiments, the inventors observed that treatment of naive N2a cells with soluble recombinant full-length Negr1 protein significantly reduced N2a growth.

The inventors also conducted a series of experiments aimed at identifying the minimal amino acid sequence capable of exerting antineoplastic activity. The Negr1 protein includes three IgG-like domains. The amino acid sequence of human full-length Negr1 protein is available from UniProtKB under accession number Q7Z3B1 (NEGR1_HUMAN) and is designated as SEQ ID NO: 1 in the appended sequence listing. Thus, the inventors cloned and characterized every single IgG-like domain. The three Negr1 IgG-like domains are hereinafter called PepA (SEQ ID NO:8), PepB (SEQ ID NO: 16) and PepC (SEQ ID NO:17). Upon treatment of naive N2a cells with Negr1, PepA, PepB, PepC, or GFP (Green fluorescent protein) as a control (GFP is approximately the same size as full-length Negr1), the inventors observed that Negr1 and PepA significantly reduced N2a proliferation, while PepB and PepC did not. The inventors also tested the ability of Negr1 and PepA to halt the growth of other cancer cell lines, namely U87 (glioblastoma), SK-Mel5 (melanoma), MCF7 (breast adenocarcinoma), A549 (adenocarcinoma alveolar basal epithelial cells), and SH-SY5Y (neuroblastoma). Treatment with rNegr1 and PepA significantly reduced the growth of all analyzed cancer lines.

The antitumour activity of PepA was also confirmed in in vivo experiments with 6-weeks old CD1 nude mice, where the inventors observed a robust reduction in tumor growth upon orthotopic PepA treatment. Additionally, the inventors found that, upon in situ administration, PepA was present in the tumor, liver and kidney, while the GFP protein used as a control was mainly in the tumor. This suggests that PepA is able to reach the blood stream and distribute into the body. Furthermore, the inventors analyzed tumor neo-vascularization by detecting CD34 protein and observed that PepA treated tumors presented a strongly reduced vascularization.

To further narrow down the minimal biologically active sequence of PepA, the inventors run an in silico analysis of the PepA structure. They cloned and characterized three partially overlapping peptides spanning the entire amino acid sequence of PepA, which are hereinafter called PepA-1 (SEQ ID NO:9), PepA-2 (SEQ ID NO:10) and PepA-3 (SEQ ID NO:11). Upon treatment of N2a cells with GFP (control), PepA, PepA-1, PepA-2 or PepA-3, the inventors observed that all the tested Negr1-derived peptides significantly reduced N2a growth, although PepA-1 and PepA-3 did so more strongly. Additionally, in a MTT assay, the inventors observed that rNegr1, PepA-1 and Lorlatinib (an FDA approved ALK inhibitor) impaired N2A growth to a similar extent. Noteworthy, the inventors further observed that treatment with PepA-1 at increasing concentrations did not elicit higher toxicity in primary cortical neurons as compared to treatment with GFP. The significant antineoplastic activity of PepA-1 was also confirmed in vivo in 6-weeks old nude mice.

Since biological evidence suggests that the human Negr1 protein is glycosylated at a position corresponding to the +10 position in the amino acid sequence of PepA-1, the inventors produced a PEGylated version of PepA-1 and verified that it possesses antitumor activity, both in vitro on N2a cells and in vivo in 6-weeks old CD1 nude mice.

The inventors also clarified the molecular mechanism underlying the antitumor activity of the Negr1-derived peptides of the invention, by identifying it in the ALK-MYCN pathway. The inventors observed that treatment of N2a cells with PepA or PepA-1 causes a reduction in ALK receptor and MYCN transcription factor protein expression levels, as well as a reduction in ERK and AKT kinases phosphorylation.

Importantly, the inventors also identified the Negr1 minimum biologically active sequence that is required for antitumor activity, i.e. GDKWSV (SEQ ID NO:6), which is present in all Negr1-derived peptides that were shown to be biologically active, namely PepA, PepA-1, PepA-2 and PepA-3, but absent from PepB and PepC, which were shown to be inactive.

Based on the above experimental results, it may be concluded that not only the Negr1 peptide of SEQ ID NO:8 (PepA), but also its fragments of at least 6 amino acids in length that contain the minimum biologically active sequence SEQ ID NO:86 located between positions 45 and 50 of SEQ ID NO:8, possess antitumor activity.

Illustrative but not limiting examples of such peptide fragments are PepA-1 (SEQ ID NO:9), PepA-2 (SEQ ID NO:10), and PepA-3 (SEQ ID NO:11). A further illustrative example is the hexapeptide consisting of the amino acid sequence SEQ ID NO:6.

The results obtained by the inventors also indicate that functional variants of the Negr1 peptides as defined above, in which the minimum biologically active sequence SEQ ID NO: 6 is replaced with SEQ ID NO:7, also possess an antitumor activity.

Illustrative but not limiting examples of such variant peptide fragments are PepA-1v (SEQ ID NO: 13), PepA-2v (SEQ ID NO:14), and PepA-3v (SEQ ID NO:15). A further illustrative example is the hexapeptide consisting of the amino acid sequence SEQ ID NO:7.

All Negr1 fragments of the invention can be provided in a pegylated form, which mimics the glycosylated form of the human Negr1 protein as it naturally occurs.

Furthermore, both the Negr1 protein and its biologically active fragments, peptide or polypeptides may be provided in a modified form, so as to improve their pharmacokinetic properties, for example to increase potency, prolong activity and/or increase half-life Such modifications may include, by way of example, glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques. Specific, illustrative examples of such modifications known per se to the person skilled in the art, which may be used in context of the present invention, include:
cyclic peptides as disclosed in Kong and Heinis 2021;
inclusion of at least two D-aminoacids as disclosed in Vlieghe et al. 2010;
inclusion of at least two beta-aminoacids as disclosed in Hook et al. 2005;
N-methylation as disclosed in Linde et al. 2008;
inclusion of different PEG groups as disclosed in Däpp et al. 2012; and/or
substitution of at least two amides in the backbone of a peptide with sulfonamides as disclosed in de Bont et al. 1999.

The following experimental section is provided by way of illustration only and is not intended to limit the scope of the invention as defined by the appended claims.

### EXPERIMENTAL SECTION

### Materials and Methods

### Constructs and Peptides

mNegr1 cDNA (Addgene clone C3342IRCKp5014P057-rzpdm13-21) was cloned into strep-FLAG pcDNA3.1 vector. mNegr1-peptides were subcloned into strep-FLAG pcDNA3.1 vector.

Scramble:
(SEQ ID NO: 18)
DGGSKGGFADLSSIIWARWNKARVDPLVSISTS
Scramble-GDKISV:
(SEQ ID NO: 19)
NRGDKISVSVSSSSGGRAWDWDIAKPGLAILTF
PepA-1-6 aa scramble:
(SEQ ID NO: 20)
DGASKGAWLNRSSIIFAGVWGSKDDPRVSISTL

### Cell Cultures and Transfection

N2a (Neuro2a, ATCC CCL-131), HEK293 (ATCC CRL-1573), U87 MG (ATCC® HTB14), SKMEL5 (ATCC® HTB70), MCF7 ATCC® HTB-22, A549 ATCC® CCL-185, and SHSY5Y (ATCC® CRL 2266™) cells were grown in DMEM high glucose (Gibco) with 10% FBS, 1% penicillin/streptomycin and 1% glutamine in a humidified atmosphere of 5% CO₂ at 37° C. Cortical neuron cultures were prepared from mouse embryos (E17.5-18.5; strain C57BL/6).

High-density (750-1000 cells/mm²) neuronal cultures were plated and grown on into 24-well plastic tissue culture plates as previously described (Iwaki; Bibby Sterilin) (Pischedda et al. 2018).

N2a and HEK293 cells were transfected with the different constructs for 48 hours using Lipofectamine 2000 (Invitrogen). Stable N2a clones expressing mNegr1 were isolated upon Neomycin selection (1 mg/ml).

### Purification on STREP-Resin

HEK293 cells transfected with the relative construct were lysed in RIPA buffer (150 mM NaCl, 50 mM HEPES, 0.5% NP40, 1% Sodium-deoxycholate) for one hour at 4° C. and then processed for streptavidin immunoprecipitation. Proteins were eluted from STREP resin in elution buffer (2.5 mM Desthiobiotin, 100 mM Tris-HCL, 150 mM NaCl, 1 mM EDTA) in mild agitation for one hour at 4° C. The protein concentration was measured via standard Bradford assay (Bio-Rad), and protein purity was assessed by SDS-PAGE followed by silver staining. Synthetic peptides were purchased at Genscript Nederland. Mice tissues were lysed in RIPA buffer (150 mM NaCl, 50 mM HEPES, 0.5% NP40, 1% Sodium-deoxycholate, 1 ml/mg of tissue) for one hour at 4° C. and then processed for streptavidin immunoprecipitation. Proteins were eluted from STREP resin in Laemmli buffer.

### MTT Assay

The 3-(4,5-dimethylthiazol-2-yl)-2,5-diphenyl tetrazolium bromide (MTT) assay was performed to evaluate culture vitality. N2a cells were cultured in a 96-well plate at a concentration of 5×10³ cell/cm². Treatment condition is indicated in the result section. At the end of the treatment, the MTT solution was added to the cell medium at a final concentration of 0.25 mg/mL. The incubation lasted 30 minutes at 37° C. Next, the medium was removed and the formazan precipitates were collected in DMSO (200 μL). The absorbance was measured at 570 nm using a spectrophotometer (Varioskan LUX, Thermofisher) as a proxy for cell viability. Relative cell viability fold is expressed over the control condition.

### Western Blotting

Protein level and relative phosphorylation was assessed by western blotting. Briefly, upon a wash in PBS, cells were solubilized in lysis buffer (150 mM NaCl, 50 mM HEPES, 0.5% NP40, 1% sodium-deoxycholate). After 1 hour under mild agitation, the lysate was clarified by centrifugation for 20 min at 16,000 g. All experimental procedures were performed at 4° C. Protein concentrations were evaluated via Bradford assay (Bio-Rad, USA). For Western blotting experiments, an equal amount of proteins was diluted with 0.25% 5× Laemmli buffer. The samples were separated on 10% SDS-PAGE gels which were transferred onto nitrocellulose membrane (Sigma-Aldrich) at 80 V for 120 min at 4° C. Primary antibodies were: Beta-actin (Santa Cruz Biotechnology, sc-47778), ALK (Santa Cruz Biotechnology, sc-398791), N-myc (Santa Cruz Biotechnology, sc-56729), Phospho-p44/42 MAPK (Erk1/2) (Thr202/Tyr204) (Cell Signaling Technology), p44/42 MAPK (Erk1/2) (137F5) Rabbit mAb (Cell Signaling Technology), Akt (pan) (11E7) Rabbit mAb (Cell Signaling Technology), Phospho-Akt (Ser473) (D9E) XP® Rabbit mAb (Cell Signaling Technology), S6 Ribosomal Protein (5G10) Rabbit mAb (Cell Signaling Technology). Antibodies were applied overnight in blocking buffer (20 mM Tris, pH 7.4, 150 mM NaCl, 0.1% Tween 20, and 5% nonfat dry milk). Proteins were detected using the ECL prime detection system (GE Healthcare). The ECL signals were acquired with the imaging ChemiDoc Touch system (Bio Rad Laboratory Italy, Segrate, Italy). The optical density of the specific bands was quantified with ImageLab software (Bio Rad).

### Animals

1 million N2a cells were suspended in 150 μl of DMEM high glucose and delivered subcutaneously above the left rear paw in 8-week old nu/nu mice (Charles River) by injection with a micro-syringe (Hamilton). All procedures involving animals were approved by Institutional and National Agencies (authorization 559/2016-PR).

### Immunofluorescence

Tumors were dissected, post-fixed for 2 hours by immersion in 4% PFA, then included in OCT and stored at −80° C. until processing. 14 μm sections were obtained after serial sectioning on Leica cryostat and mounted on Polysine Slides (Thermo Fischer Scientific). The slices were saturated in 2.5% BSA, 10% NGS, 0.2% Triton X-100, PBS 1×, for 1 hour at RT and incubated with the primary antibody O/N at 4° C. (Anti-CD34 antibody [EP373Y], ABCAM, ab81289). After 3 washes in PBS 1×-0.2% Triton X-100 the sections were incubated for 1 h with the conjugated secondary antibody at RT (AffiniPure Goat Anti-Rabbit IgG (H+L) Alexa Fluor 594, Jackson ImmunoResearch, 111-585-003). The slices were then rinsed 3 times in PBS 1×-0.2% Triton X-100, rinsed once in PBS1× and then mounted in water-based mounting media. Sections were kept at 4° C. in the dark until acquisition with a Zeiss Axio Imager M2 equipped with 40× objective.

### Statistical Analysis and Guidelines

All data were plotted as box, with minimum, maximum, and median indicated. The normality of data distributions was determined using the D'Agostino and Pearson omnibus normality test, followed by an unpaired Student's t test, ANOVA followed by Tuckey's post-hoc test or two-way ANOVA followed by Bonferroni or Student's t post-hoc test as appropriate. The number of experiments (n) and level of significance (p) are indicated throughout the text. All methods were performed in accordance with the relevant guidelines and national regulations.

### Results

### Negr1 Over-Expression Halts Cancer Growth In Vitro and In Vivo

Protein samples obtained from human neuroblastoma (SH5Y), glioblastoma (U87), murine neuroblastoma (N2a) were processed by western-blotting. The inventors observed that the Negr1 protein is downregulated in the brain cancer-derived lines. In contrast, Negr1 could be detected in terminally differentiated murine cortical neurons (FIG. 1A). Next, the inventors assessed whether the Negr1 protein levels correlated with cellular proliferation. To this aim, several N2a clones expressing Strep-FLAG Negr1 fusion protein were generated and characterized. The N2a clones were seeded at a fixed amount (20,000 cells) at day in vitro 0 (DIV0). At DIV5, cellular proliferation was evaluated. It was found that ectopic Negr1 expression significantly reduced N2a proliferation (FIG. 1B).

It is known that cancer cells are able to grow independently of a solid surface. Such anchorage-independent growth is a hallmark of carcinogenesis. This feature can be recapitulated in vitro by monitoring colony growth within a soft agar matrix. Thus, the soft agar colony formation assay tests whether cells have undergone a malignant transformation. Accordingly, a fixed amount (20,000 cells) of wild-type and Negr1-expressing cells was seeded in soft-agar and cultured for 14 days. Next, cells were visualized by the crystal violet staining. By scoring the number of the colonies (cluster encompassing more than 50 cells), the inventors noticed that Negr1-expressing N2a cellular clones had a severely reduced ability to generate colonies on soft agar (FIG. 1C-D).

Xenograft mouse models represent an established tool to investigate cancer progression in vivo. To this aim, 1 million N2a cells (naive, clone 3, and clone 11) was injected subcutaneously (s.c.) in immunodeficient CD1 nude mice. Tumor volume was monitored ex-vivo by measuring tumor size with a Vernier caliper. Tumor volume was calculated using the ellipsoid formula (length×width×height×0.52). The inventors noticed that the tumors derived from Negr1 expressing cells were significantly smaller in size than the tumors derived from wild-type N2a cells (FIG. 1E-F).

### Soluble Negr1 Halts Cancer Growth In Vitro and In Vivo

At the physiological level, Negr1 exists as a GPI-anchored membrane-bound protein and as a soluble protein released upon metalloprotease cleavage (Sanz et al. 2015; Pischedda and Piccoli 2015). Thus, the inventors studied whether ectopic treatment with soluble recombinant Negr1 (Negr1) might modulate cancer cell growth. To this aim, the inventors expressed and purified, from HEK293 cells, Strep-FLAG Negr1 and Strep-GFP, the latter one being a biologically inert protein used as control. Next, 10,000 naive N2a cells were seeded and treated daily with 200 ng rNegr1 or GFP. At DIV5, cellular growth was evaluated by MTT assay. The inventors observed that treatment with Negr1 significantly reduced N2a growth (FIG. 2A).

Next, the inventors aimed at identifying the minimal sequence capable of exerting Negr1-antineoplastic action. The Negr1 protein encompasses three IgG-like domains. Thus, the inventors cloned and characterized every single IgG-like domain as a strep-FLAG tagged recombinant protein. The inventors expressed and purified, from HEK293 cells, the three domains, hereinafter called PepA, PepB, PepC.

Next, 10,000 naive N2a cells were seeded and treated daily with 200 ng Negr1, PepA, PepB, PepC, or GFP. At DIV5, cell number was evaluated. The inventors observed that treatment with Negr1 and PepA significantly reduced N2a proliferation (FIG. 2B).

Next, the capability of Negr1 and peptide PepA to halt the growth of human cancer-derived cell lines, namely U87 (glioblastoma), SK-Mel5 (melanoma), MCF7 (breast adenocarcinoma), A549 (adenocarcinoma alveolar basal epithelial cells), and SH-SY5Y (neuroblastoma) was tested. To this aim, each line was treated daily with 200 ng GFP, rNegr1, or PepA. At DIV5, cellular growth was evaluated by MTT assay. Treatment with rNegr1 and PepA was found to significantly reduce the growth of all tested cancer lines (FIG. 2C). Such results are in good agreement with the expression of the receptor ALK, that has been experimentally detected in cancer cell lines of ectodermal origin (including small cell lung carcinoma, breast carcinoma, melanoma, neuroblastoma, and glioblastoma) (Dirks et al. 2002).

To further assess the anti-neoplastic activity of PepA, 1 million N2a cells were injected s.c in 6-weeks old CD1 nude mice. When the tumor reached 100 mm³ volume, 2 μg of GFP or PepA were injected in situ. The treatment was repeated every second day, and tumor growth was monitored daily. A robust growth reduction in tumor was observed upon PepA treatment (FIG. 3A).

At day 9 post injection (or earlier in case the tumor reached 2.5 cm³ volume), the animals were sacrificed and the mice tissues were harvested and analysed for the presence of GFP and PepA. In particular, upon immobilization on a STREP-resin, the inventors observed that PepA was present in the tumor, liver, and kidney, while the GFP protein was mainly in the tumor (FIG. 3B). This biochemical evidence suggests that PepA is able to reach the bloodstream and distribute into the body.

Cancer growth requires and triggers vascularization. In particular, cancer mass is characterized by the presence of immature vessels decorated by CD34 protein. Thus, the inventors analyzed tumor neo-vascularization by detecting CD34 protein. PepA treated tumors presented a strongly reduced vascularization in terms of total area stained by CD34 and average vessel area (FIG. 3C-E).

### Characterization of a Biological 33-Aminoacids Long Anti-Neoplastic Peptide

To further narrow down the minimal biologically active sequence within PepA, the PepA structure was analysed in silico. In agreement with the predicted secondary structures, the inventors cloned and characterized three peptides covering PepA sequence (FIG. 4A). The three peptides were expressed and purified from HEK293 cells as strep-FLAG tagged recombinant peptides, hereinafter called PepA-1 (33 aminoacids), PepA-2 (70 aminoacids), PepA-3 (70 aminoacids). To assess the in vitro activity of the three peptides, 10,000 naive N2a cells were seeded and treated daily with 500 ng/ml GFP, PepA, PepA-1, PepA-2 and PepA-3. At DIV5, cellular growth was evaluated by MTT assay. The inventors observed that the minimal peptide PepA-1 reduced significantly N2a growth (FIG. 4B).

To further examine the efficacy of PepA1, N2A cell proliferation was investigated upon chronic treatment with vehicle, Lorlatinib (an FDA approved ALK inhibitor), GFP, rNegr1, and PepA-1, (daily, all molecules at 3 μM). At DIV5, cellular vitality was evaluated by MTT assay. rNegr1, PepA-1 and Lorlatinib impaired N2A growth to a similar extent (FIG. 4C). Interestingly, treatment with PepA-1 at increasing concentration (daily, from DIV7 to 12) did not elicit over toxicity on DIV12 primary cortical neurons if compared to GFP treatment (FIG. 4D).

Next, 1 million N2a cells were injected s.c in 6-weeks old nude mice. When tumor reached 100 mm³ volume, 2 ug of GFP or PepA-1 were injected in situ. The treatment was repeated every second day and the tumor growth was monitored daily. The inventors observed a robust growth reduction upon PepA-1 treatment (FIG. 4E).

### Characterization of a Synthetic 33-Aminoacids Long Anti-Neoplastic Peptide

Biochemical evidence suggests that PepA1 is glycosylated on the asparagine residue (N) in position+10. The inventors designed a synthetic peptide encompassing PepA-1 sequence coupled at the N-terminus to a mini-PEG1 moiety (MW: 152 Da) to mimic glycosylation. The resulting peptide, hereinafter called PEG-PepA-1, is soluble in water at a concentration of 5 mg/ml.

To evaluate the anti-neoplastic activity of PEG-PepA-1, 1 million N2a cells were injected s.c in 6-weeks old CD1 nude mice. When tumor reached 100 mm³ volume, 10 μg of PEG-PepA-1 or PEG-scramble peptide were injected in situ. Treatment was repeated every second day and tumor growth was monitored daily. The inventors observed a robust growth reduction in tumor upon PepA treatment (FIG. 5A).

Finally, the inventors tested the efficacy of PEG-PepA-1 upon systemic administration. To this aim, 1 million N2a cells were injected s.c in 6-weeks old CD1 nude mice. When tumor reached 100 mm³ volume, 150 μg of PEG-PepA-1 or PEG-scramble peptide were injected intraperitoneally. The treatment was repeated every second day and the tumor growth was monitored daily. The inventors observed a robust growth reduction in tumor upon PEG-PepA-1 treatment (FIG. 5B).

Given the robust effect observed on cancer cell growth in vitro and in vivo, the inventors sought to understand the molecular mechanisms underlining the anti-neoplastic effect elicited by Negr1 derived peptides (FIG. 6A). Altered activity of ALK receptor and genetic amplification and therefore increased expression of the downstream MYCN transcription factor are the pivotal molecular hallmarks of neuroblastoma. In addition, the ALK gene can be oncogenically altered in several malignancies, including non-small-cell lung cancer (NSCLC) and anaplastic large cell lymphomas (ALCL).

The inventors monitored whether treatment with rNegr1 or PepA might impact on ALK-MYCN pathway. To this aim, 50,000 N2a cells were treated with 300 ng/ml GFP, rNegr1 or PepA daily for 5 days. At DIV5, the culture was processed for western blotting and ALK and MYCN protein expression levels were evaluated. PepA treatment induced a robust reduction of ALK and MYCN protein levels (FIG. 6B-D).

Next, 50,000 N2a cells were treated with 3 μM GFP, Lorlatinib or PepA-1 (50 ng/ml) daily for 5 days. At DIV5, the culture was processed for western-blotting, and ALK and MYCN protein expression was evaluated. Noteworthy, the inventors noticed that the chronic treatment with PepA-1 triggered a strong down-regulation of ALK and MYC-N (FIG. 6E-G).

ALK triggers a signaling cascade that involves ERK and AKT kinases phosphorylation. Thus, in order to complement the above-illustrated findings, the inventors evaluated the impact of PepA-1 on ERK and AKT phosphorylation. Upon acute treatment (3 μM, 10 minutes), PepA-1 significantly reduced ERK and AKT phosphorylation (FIG. 6H-J).

Additionally, the N2a cells were treated with the following synthetic peptides: the synthetic version of PepA-1 (synPepA-1), a scramble peptide including the GDKISV sequence (scr-GDKISV, substituting the W, tryptophan, that can undergo oxidation, with I, Isoleucine), and a scramble peptide (10 μM, 10 minutes). Noteworthy, the inventors found that scr-GDKISV and synPepA-1 reduced ERK1/2 phosphorylation (FIG. 7C-D).

In a complementary approach, N2a cells were treated with a PepA-1-derived peptide where the GDKWSV sequence was replaced by a scramble sequence (PepA-1-6aa scramble, 10 μM, 10 minutes). The inventors observed that PepA-1-6aa scramble did not alter ERK phosphorylation (FIG. 7E-F).

Finally, the biological activity of the GDKWSV peptide alone was tested. To this aim, 150,000 N2a cells were treated with a synthetic GDKWSV peptide or with the ALK inhibitor Lorlatinib (30 nM, 10 minutes). By dot-blot, the inventors observed that GDKWSV peptide treatment correlates with a severe reduction of ERK1/2 phosphorylation (FIG. 7E-F). This finding suggests that the esapeptide GDKWSV is crucial to sustaining the biological activity of the PepA-1 peptide. Eventually, the inventors monitored the pharmacodynamic properties of PEG-PepA1. Upon acute treatment (30 μM, 10 minutes), PEG-PepA-1 significantly reduced ERK phosphorylation (FIG. 8A-B).

The inventors also studied the effect of the combination of PEG-PepA-1 with lorlatinib. They observed that, upon long-term treatment, PEG-PepA-1 significantly reduced the level of ALK protein, while lorlatinib induced a robust up-regulation of the receptor (both at 5 μM, 24 or 48 hours, FIG. 8C). Noteworthy, the co-administration of PEG-PepA-1 significantly reduced the level of ALK protein noticed upon long-term treatment with lorlatinib (5 μM, 24 hours, FIG. 8D).

### REFERENCES

Bhoopathi P., Mannangatti P., Emdad L., Das S. K., Fisher P. B. (2021) The quest to develop an effective therapy for neuroblastoma. J Cell Physiol.
Bobyn A., Zarrei M., Zhu Y., Hoffman M., Brenner D., Resnick A. C., Scherer S. W., Gallo M. (2020) Ancestry and frequency of genetic variants in the general population are confounders in the characterization of germline variants linked to cancer. BMC Med Genet 21, 92.
Bont D. B. A. de, Sliedregt-Bol K. M., Hofmeyer L. J. F., Liskamp R. M. J. (1999) Increased stability of peptidesulfonamide peptidomimetics towards protease catalyzed degradation. Bioorganic & Medicinal Chemistry 7, 1043-1047.
Däpp S., Müller C., Garayoa E. G., Bläuenstein P., Maes V., Brans L., Tourwé D. A., Schibli R. (2012) PEGylation, increasing specific activity and multiple dosing as strategies to improve the risk-benefit profile of targeted radionuclide therapy with 177Lu-DOTA-bombesin analogues. EJNMMI Research 2, 24.
Dirks W. G., Fähnrich S., Lis Y., Becker E., MacLeod R. A. F., Drexler H. G. (2002) Expression and functional analysis of the anaplastic lymphoma kinase (ALK) gene in tumor cell lines. Int J Cancer 100, 49-56.
Hasan M. K., Nafady A., Takatori A., Kishida S., Ohira M., Suenaga Y., Hossain S., et al. (2013) ALK is a MYCN target gene and regulates cell migration and invasion in neuroblastoma. Sci Rep 3, 3450.
Hook D. F., Bindschädler P., Mahajan Y. R., Šebesta R., Kast P., Seebach D. (2005)
The Proteolytic Stability of ‘Designed’ β-Peptides Containing α-Peptide-Bond Mimics and of Mixed α,β-Peptides: Application to the Construction of MHC-Binding Peptides. Chemistry & Biodiversity 2, 591-632.
Janoueix-Lerosey I., Lequin D., Brugières L., Ribeiro A., Pontual L. de, Combaret V., Raynal V., et al. (2008) Somatic and germline activating mutations of the ALK kinase receptor in neuroblastoma. Nature 455, 967-970.
Kim H., Hwang J. S., Lee B., Hong J., Lee S. (2014) Newly Identified Cancer-Associated Role of Human Neuronal Growth Regulator 1 (NEGR1). J Cancer 5, 598-608.
Kong X. D., Heinis C. (2021) Towards the Development of Orally Available Peptide Therapeutics. Chimia (Aarau) 75, 514-517.
Linde Y., Ovadia O., Safrai E., Xiang Z., Portillo F. P., Shalev D. E., Haskell-Luevano C., Hoffman A., Gilon C. (2008) Structure-activity relationship and metabolic stability studies of backbone cyclization and N-methylation of melanocortin peptides. Peptide Science 90, 671-682.
Mahapatra S., Challagundla K. B. (2021) Neuroblastoma, in StatPearls. StatPearls Publishing, Treasure Island (FL).
Mckie A. B., Vaughan S., Zanini E., Okon I. S., Louis L., Sousa C. de, Greene M. I., et al. (2012) The OPCML tumor suppressor functions as a cell surface repressor-adaptor, negatively regulating receptor tyrosine kinases in epithelial ovarian cancer. Cancer Discov 2, 156-171.
Miyata S., Matsumoto N., Taguchi K., Akagi A., lino T., Funatsu N., Maekawa S. (2003) Biochemical and ultrastructural analyses of IgLON cell adhesion molecules, Kilon and OBCAM in the rat brain. Neuroscience 117, 645-658.
Park J, Choi H, Kim Y D, Kim S H, Kim Y, Gwon Y, Lee D Y, Park S H, Heo W D, Jung Y K. Aberrant role of ALK in tau proteinopathy through autophagosomal dysregulation. Mol Psychiatry. 2021 Jan. 15.
Pischedda F., Montani C., Obergasteiger J., Frapporti G., Corti C., Rosato Siri M., Volta M., Piccoli G. (2018) Cryopreservation of Primary Mouse Neurons: The Benefit of Neurostore Cryoprotective Medium. Front Cell Neurosci 12, 81.
Pischedda F., Piccoli G. (2015) The IgLON Family Member Negr1 Promotes Neuronal Arborization Acting as Soluble Factor via FGFR2. Front Mol Neurosci 8, 89.
Pischedda F., Szczurkowska J., Cirnaru M. D., Giesert F., Vezzoli E., Ueffing M., Sala C., et al. (2014) A cell surface biotinylation assay to reveal membrane-associated neuronal cues: Negr1 regulates dendritic arborization. Mol. Cell Proteomics 13, 733-748.
Sanz R., Ferraro G. B., Fournier A. E. (2015) IgLON Cell Adhesion Molecules Are Shed from the Cell Surface of Cortical Neurons to Promote Neuronal Growth. J. Biol. Chem. 290, 4330-4342.
Schönherr C., Ruuth K., Kamaraj S., Wang C. L., Yang H. L., Combaret V., Djos A., et al. (2012) Anaplastic Lymphoma Kinase (ALK) regulates initiation of transcription of MYCN in neuroblastoma cells. Oncogene 31, 5193-5200.
Speleman F., Park J. R., Henderson T. O. (2016) Neuroblastoma: A Tough Nut to Crack. Am Soc Clin Oncol Educ Book 35, e548-557.
Szczurkowska J., Pischedda F., Pinto B., Managò F., Haas C. A., Summa M., Bertorelli R., et al. (2018) NEGR1 and FGFR2 cooperatively regulate cortical development and core behaviours related to autism disorders in mice. Brain.
Takita J., Chen Y., Okubo J., Sanada M., Adachi M., Ohki K., Nishimura R., et al. (2011) Aberrations of NEGR1 on 1p31 and MYEOV on 11q13 in neuroblastoma. Cancer Sci. 102, 1645-1650.
Villa M, Sharma G G, Manfroni C, Cortinovis D, Mologni L. New Advances in Liquid Biopsy Technologies for Anaplastic Lymphoma Kinase (ALK)—Positive Cancer. Cancers. 2021; 13(20):5149.
Vlieghe P., Lisowski V., Martinez J., Khrestchatisky M. (2010) Synthetic therapeutic peptides: science and market. Drug Discov Today 15, 40-56.

1. Method of therapeutically treating a disease selected from a tumor disease and a tauopathy in a patient in need thereof with an isolated Negr1 protein or a biologically active fragment thereof of at least 6 amino acids in length capable of reducing ALK protein expression levels, said method comprising
administering to said patient a therapeutically effective amount of said Negr1 protein or of said biologically active fragment thereof of at least 6 amino acids in length.

2. The method according to claim 1, wherein the isolated Negr1 protein is selected from the group consisting of:
(i) amino acid sequences which comprise SEQ ID NO: 1, SEQ ID NO:2, SEQ ID NO:3, SEQ ID NO:4 or SEQ ID NO:5;
(ii) amino acid sequences which comprise an amino acid sequence at least 90% identical to SEQ ID NO: 1, SEQ ID NO:2, SEQ ID NO: 3, SEQ ID NO:4 or SEQ ID NO:5 and which are capable of reducing ALK protein expression levels; and
(iii) amino acid sequences which comprise an amino acid sequence at least 90% identical to SEQ ID NO: 1, SEQ ID NO:2, SEQ ID NO: 3, SEQ ID NO:4 or SEQ ID NO:5 and which include the minimum biologically active sequence SEQ ID NO:6 or SEQ ID NO:7.

3. The method according to claim 2, wherein the biologically active fragment includes the minimum biologically active sequence SEQ ID NO:6 or SEQ ID NO: 7.

4. The method according to claim 1, wherein the biologically active fragment is selected from the group consisting of SEQ ID NO:6, SEQ ID NO:7, SEQ ID NO: 8, SEQ ID NO:9, SEQ ID NO: 10, SEQ ID NO: 11, SEQ ID NO: 12, SEQ ID NO:
13, SEQ ID NO: 14 and SEQ ID NO: 15.

5. The method according to claim 1, which includes a stability-increasing modification, wherein the stability-increasing modification is selected from the group consisting of N-terminal modifications, C-terminal modifications, side chain modifications, amino acid modifications, backbone modifications, and any combination thereof, or
wherein the stability-increasing modification is selected from the group consisting of PEGylation, cyclization, N-methylation, replacement of at least two amino acid residues with the corresponding D-stereoisomers, replacement of at least two amides in the backbone with sulfonamides, and any combination thereof.

6.-7. (canceled)

8. The method according to claim 1, wherein the disease is a tumor disease and wherein said administering step comprises a combined simultaneous, separate or sequential administration of a compound or substance having anti-tumor activity or with a radiotherapy agent.

9. The method according to claim 8, wherein the compound or substance having anti-tumor activity is selected from the group consisting of Cyclophosphamide, Cisplatin, carboplatin, Vincristine, Doxorubicin, Etoposide, Topotecan, Melphalan, Busulfan, Thiotepa, dinutuximab, dinutuximab beta, Crizotinib, Ceritinib, Alectinib, Brigatinib, Lorlatinib, Ensartinib, Entrectinib, and any combination thereof.

10. The method according to claim 1, wherein the tumor disease is selected from the group consisting of neuroblastoma, ganglioglioma, gangliocytoma, adenocarcinoma of the lung, renal cell carcinoma, squamous cell esophageal carcinoma, breast cancer, thyroid cancer, colon adenocarcinoma, glioblastoma, squamous cell esophageal carcinoma, melanoma, ovarian cancer, non-small cell lung cancer (NSCLC), anaplastic large-cell lymphoma (ALCL) and inflammatory myofibroblastic tumor (IMT).

11. The method according to claim 1, wherein the disease is a tauopathy and wherein said administering step comprises use is a combined simultaneous, separate or sequential administration of a compound or substance active in the therapeutic treatment of the tauopathy.

12. The method according to claim 1, wherein the tauopathy is selected from the group consisting of Alzheimer's disease, postencephalitic parkinsonism, Chronic traumatic encephalopathy (CTE), Parkinson-dementia complex of Guam, Frontotemporal dementia and parkinsonism linked to chromosome 17 (FTDP-17), Primary age-related tauopathy (PART) dementia, Progressive supranuclear palsy (PSP), Corticobasal degeneration (CBD), Vacuolar tauopathy, Meningioangiomatosis, Subacute sclerosing panencephalitis (SSPE).

13. An isolated peptide or polypeptide of at least 6 amino acids in length, which is a fragment of a Negr1 protein and is capable of reducing ALK protein expression levels, wherein said fragment is selected from the group consisting of:
(i) amino acid sequences which comprise SEQ ID NO: 1, SEQ ID NO:2, SEQ ID NO: 3, SEQ ID NO:4 or SEQ ID NO:5; and
(ii) amino acid sequences which comprise an amino acid sequence at least 90% identical to SEQ ID NO: 1, SEQ ID NO:2, SEQ ID NO:3, SEQ ID NO:4 or SEQ ID NO: 5.

14. (canceled)

15. The isolated peptide or polypeptide according to claim 13, which includes the minimum biologically active sequence SEQ ID NO:6 or SEQ ID NO:7.

16. The isolated peptide or polypeptide according to claim 15, which is selected from the group consisting of SEQ ID NO: 6, SEQ ID NO: 7, SEQ ID NO: 8, SEQ ID NOV, SEQ ID NO: 10, SEQ ID NO: 11, SEQ ID NO: 12, SEQ ID NO: 13, SEQ ID NO: 14 and SEQ ID NO:15.

17. The isolated peptide or polypeptide according to claim 13, which includes a stability-increasing modification, wherein the stability-increasing modification is selected from the group consisting of N-terminal modifications, C-terminal modifications, side chain modifications, amino acid modifications, backbone modifications, and any combination thereof or wherein the stability-increasing modification is selected from the group consisting of PEGylation, cyclization, N-methylation, replacement of at least two amino acid residues with the corresponding D-stereoisomers, replacement of at least two amides in the peptide backbone with sulfonamides, and any combination thereof.

18.-23. (canceled)

24. A pharmaceutical composition comprising
an isolated peptide or polypeptide according to claim 13 or
an isolated nucleic acid coding for the Negr1 protein or for a protein or for a polypeptide of at least 6 amino acids in length capable of reducing ALK protein expression levels and pharmaceutically acceptable excipient.

25. (canceled)
